(19) United States
(12) Patent Application Publication
(10) Pub. No.: US 2006/0155154 A1
(43) Pub. Date: Jul. 13, 2006

(54) Process for the selective hydrogenation of alkynes

(75) Inventors: J. Yong Ryu, League City, TX (US); Christopher C. Boyer, Houston, TX (US)

Correspondence Address: KENNETH H. JOHNSON, P.O. BOX 630708, HOUSTON, TX, 77263, US

(73) Assignee: CATALYTIC DISTILLATION TECHNOLOGIES

(21) Appl. No.: 11/031,900

(22) Filed: Jan. 7, 2005

(51) Int. Cl. C07C 5/03

(52) U.S. Cl. 585/259

(57) ABSTRACT

A process for removal of acetylenic compounds from hydrocarbon streams in which a hydrocarbon feed having a target fraction which contains a first concentration of acetylenic compounds and olefins is contacted with a catalyst selective for the hydrogenation of acetylenic compounds in the presence of hydrogen and a solvent having a boiling point higher than the boiling point of the target fraction in a distillation reaction zone under conditions of temperature, pressure and hydrogen concentration favoring the hydrogenation of acetylenic compounds in which the target fraction is recovered as overheads having a second concentration of acetylenic compounds lower than said first concentration and the solvent is recovered as bottoms.

### BACKGROUND OF THE INVENTION

1. Field of the Invention

The present invention relates to the selective removal of more highly unsaturated compounds from mixtures of unsaturated compounds. More particularly the invention is concerned with the selective hydrogenation of acetylenic compounds from mixtures with dienes, such as 1,3-butadiene. The invention provides a novel process for the selective hydrogenation of acetylenes in admixture with other unsaturated compounds.

2. Related Information

The crude streams for the commercial production of olefins and dienes contain various compounds as impurities. Acetylenic impurities need to be removed from the streams to produce acceptable quality olefin and diene products. A preferred technique for removing the acetylenic impurities is partial hydrogenation, often called selective hydrogenation. For the commercial production of olefins and dienes, the catalytic hydrogenation of acetylenic compounds is utilized to remove acetylenic impurities in the crude product stream.

To produce olefins such as ethylene, propylene, butadiene, isoprene and the like, acetylenic impurities such as acetylene, methyl acetylene, vinyl acetylene, ethyl acetyiene, 2-methyl-1-buten-3-yne and the like, in various crude mixed C₂-C₅ streams need to be removed with minimum loss of useful materials such as ethylene, propylene, butenes, butadiene, isoprene and the like in the feed streams. The preferred technique for the purification in commercial practice is the selective hydrogenation of acetylenic compounds over hydrogenation catalysts.

The difficulty in the catalytic hydrogenation of acetylenic compounds arises from the fact that the hydrogenation must be carried out in the presence of a large excess of olefins or dienes or both. Under industrial conditions, valuable olefin and diene products in the crude product streams are not inert. This is especially true as the conversion of acetylenic compounds approaches completion, resulting in the loss of valuable products. Therefore, during the selective hydrogenation of acetylenic compounds, minimizing the loss of olefins and dienes is highly desirable for the commercial production of olefins such as ethylene, propylene, and styrene and dienes such as 1,3-butadiene and isoprene. The selectivity of a catalyst is often the determining factor in selecting a catalyst for the production of olefins and dienes.

The difficulty of hydrogenating an acetylenic group in a molecule depends on the location of the triple bond on the molecule whether there is conjugation or an olefin group. An isolated terminal triple bond is easiest to selectively hydrogenate. A conjugated triple bond with a double bond is much more difficult for the selective hydrogenation. G. C. Bond et al., J. Catalysis 174, 1962, reported in the hydrogenation of acetylene, methyl acetylene, and dimethyl acetylene that the order of decreasing catalyst selectivity is Pd>Rh>Pt>Ru>Os>Ir. L. Kh. Freidlin et al., DokI. Akad. Nauk SSSR 152 (6), 1383, 1962, reported that the order of decreasing catalyst selectivity is palladium black>platinum black>rhodium black>Raney nickel>Raney cobalt for the terminal acetylenes and palladium black>Raney nickel>platinum black>Raney cobalt>rhodium black for internal acetylenes.

Palladium on barium sulfate is reported to be more selective than Raney nickel in hydrogenation of vinyl acetylene in liquid phase (Catalytic Hydrogenation over Platinum Metals by Paul. N. Rylander, p. 75, Academic Press, 1967). Product analysis at 100% conversion of vinyl acetylene indicates that the product from Raney nickel catalyst contains only about half the butadiene (35%) and 23 times the butane (23%) compared with the product from palladium supported on barium sulfate.

Supported Pd, Ni, Cu and Co catalysts have been known to be useful for the hydrogenation of acetylenes (Handbook of Commercial Catalysts, pp. 105-138, Howard F. Rase, CRC Press, 2000).

The most preferred catalysts in commercial application of selective hydrogenation of acetylenes are palladium-based catalysts such as Pd, Pd/Pb, Pd/Ag or Pd/Au on a support such as alumina and the copper catalyst on a support such as alumina. Pd catalysts are the most preferred catalysts because of high activity and supposedly superior selectivity compared with other metal catalysts.

The prior art widely demonstrates that palladium catalysts have the highest selectivity for the selective hydrogenation of acetylenes among Group VIII metals. No art has been found showing higher selectivity of nickel catalysts over palladium catalysts. In fact, palladium catalysts are the choice of all current commercial processes for the selective hydrogenation of acetylenic impurities (vinyl acetylene, ethyl acetylene and methyl acetylene) in crude butadiene streams and crude C₃ olefin streams.

1,3-Butadiene is an important raw material for production of various polymers such as butadiene-styrene copolymer. One of the processes for producing 1,3-butadiene is co-production of various olefins by steam cracking of petroleum fractions. The crude mixed C₄ stream from a steam cracker is selectively hydrogenated to partially remove C₄ acetylenic compounds. The selectively hydrogenated stream is sent to the 1,3-butadiene recovery unit where solvent extractive distillation is used to separate 1,3-butadiene from the rest of components in the mixed stream. Complete removal of C₄ acetylenic compounds in the stream with high recovery of 1,3-butadiene is highly desirable to reduce the production cost of 1,3-butadiene and produce premium quality product for polymer production. Heretofore, it was technically impossible to completely remove C₄ acetylenes in crude mixed streams by selective hydrogenation without an unacceptably high loss of 1,3-butadiene due to over hydrogenation of 1,3-butadiene. Therefore, an improved inexpensive process via highly active and selective catalysts is highly desirable to produce premium quality 1,3-butadiene without paying a penalty for high loss of 1,3-butadiene due to over hydrogenation.

The palladium-based catalysts for selective hydrogenation of C₄ acetylenic compounds are highly active. However, their level of selectivity does not allow complete removal of C₄ acetylenes without an unacceptable high loss of 1,3-butadiene due to over hydrogenation. Another inherent problem of palladium-based catalysts is the loss and migration of palladium due to the formation of soluble Pd complex compound by the reaction of Pd atoms on the catalyst surface with vinyl acetylene, if the hydrogenation is carried out in the presence of a liquid phase. Silver and gold have been used to minimize the loss of palladium and reduce catalytic polymerization of acetylenic compounds. Palladium-based catalysts are disclosed in U.S. Pat. No. 5,877,363 (1999), and EP 0 089 252 (1983). U.S. Pat. No. 5,877,363 (1999) disclosed the process for the selective hydrogenation of acetylenic impurities and 1,2-butadiene in mixed olefin rich C₄ streams by using supported Pt and Pd catalysts.

The copper-based catalyst is very selective so that the recovery of 1,3-butadiene from the mixed stream is higher than palladium-based catalysts. However, since the activity of copper catalysts is very low compared with palladium-based catalysts, a large volume of catalyst and large reactor are required. The copper catalyst cokes up quickly and frequent regeneration of the catalyst is necessary. Such catalysts are disclosed in U.S. Pat. No. 4,440,956 (1984) and U.S. Pat. No. 4,494,906 (1985).

The selective hydrogenation of C₃ and C₄ acetylenic compounds in a crude butadiene stream over a supported commercial Pd (0.2 wt. %)-Ag (0.1 wt. %) catalyst decreases as the hydrogenation temperature increases; noted by H. Uygur et al. in liquid phase selective hydrogenation of methyl acetylene/propadiene (MAPD) in a mixed C₃ stream (J. Chem. Eng. Japan, 31, p. 178, 1998).

According to R. S. Mann et al. (Can. J. Chem. 46, p. 623, 1968), Ni and Ni—Cu alloy catalysts are effective for methyl acetylene hydrogenation. The catalytic activity rapidly increases with the addition of copper to nickel up to 25 wt. % in alloy catalyst. The selectivity to propylene and extent of polymerization increase with increasing of copper in the alloy.

According to H. Gutmann and H. Lindlar (Organic Synthesis, Chapter 6), vinyl acetylene and 2-methyl-1-buten-3-yne are difficult to selectively hydrogenate to 1,3-butadiene and isoprene by using the usual palladium, nickel or cobalt catalysts. But the palladium catalyst supported on calcium carbonate treated with mercury acetate is useful for the selective hydrogenation.

Nickel-based catalysts are known in the art to be effective for the selective hydrogenation of acetylenic impurities in mixed streams of olefins. It is well documented that nickel catalysts in any form are highly active for hydrogenation of olefins and benzene. Because of very high activity of Ni catalysts for hydrogenation of olefins, the selective hydrogenation of acetylenes in mixtures of dienes or olefins is preferentially carried out over the presulfided nickel catalyst or in the presence of moderating agent for the nickel catalysts, as known in the prior art.

Unsulfided metallic nickel or unsulfided metallic nickel modified with metallic Mo, Re, Bi or mixtures in which the catalyst is used alone or is used in combination with other acetylenic selective catalysts are very beneficial of the selective hydrogenation of acetylenic compounds as U.S. Ser. No. 10/215,096 filed Aug. 8, 2002 and incorporated herein in its entirety.

U.S. Pat. No. 4,504,593 teaches the use of supported bimetallic catalyst comprised of at least one group VIII metal selected from the Pt, Pd, Ni and Co group, and at least one metal from the Ge, Sn, and Pb group for selective hydrogenation of acetylenic hydrocarbons and diolefins in the olefinic mixtures to mono-olefins. The catalyst contains 0.1 to 10 wt. % Ni, preferably from 1 to 5 wt. %, on a support such as alumina (70 m²/g and 0.5 cc/g total pore volume). The catalysts are prepared in two steps, introducing the second component (Ge, Sn or Pb) of the catalyst to the Ni catalyst from the first step. The selective hydrogenation is preferably carried out in the presence of sulfur and nitrogen compound to obtain acceptable improved selectivity. However, the patent does not suggest the selective hydrogenation of C₄ acetylenes in mixed butadiene streams in the absence of sulfur with the activated Ni metal catalyst.

U.S. Pat. No. 3,793,388 (1974) disclosed the selective hydrogenation of acetylene in olefin mixtures in the presence of nickel catalyst supported on alumina. The alumina is characterized by having a substantial portion of pores having at least 120 Å diameter and at least 2 m²/g surface area. The nickel content on the catalyst is from about 0.5 to about 8 mg per square meter of total alumina surface area.

Br 1,182.929 (1970) disclosed a useful catalyst for selective hydrogenation of acetylenic hydrocarbons in an olefin mixture such as crude butadiene stream. The catalyst is the nickel promoted copper catalyst supported on a carrier. The weight of the copper component on the catalyst exceeds the weight of Ni and the weight of the carrier exceeds the weight of active metal components. The final catalyst in mixed oxide form is prepared by calcining a mixture of oxides at 850° C. The catalyst is activated by reducing the temperature from 180° to 600° C. with a hydrogen-containing gas. The metallic active components on the activated catalyst is at least 25% by weight of the active metal components. The remaining percentage is in the form of their oxides. The selective hydrogenation is carried out in gas phase at a temperature from 100° to 250° C. and about 1 WHSV. The cycle time is about 420 hours.

U.S. Pat. No. 4,748,290 (1988) disclosed a nickel boride catalyst supported on alumina for hydrogenation of acetylenic and diolefinic compounds to monoolefinic compound. Reacting supported nickel arsenate with a borohydride compound activates the catalyst.

U.S. Pat. No. 4,831,200 (1989) disclosed the process for a two-step selective hydrogenation of acetylenic impurities in crude butadiene stream. The acetylenic impurities in crude feed streams are partially hydrogenated in the palladium-based catalyst disclosed in U.S. Pat. No. 4,533,779 and then the remaining impurities are hydrogenated in the copper-base catalyst disclosed in U.S. Pat. Nos. 4,493,906 and 4,440,956 discussed above.

Supported copper catalysts and palladium catalysts have been preferred catalysts in cleaning up acetylenic impurities in olefin streams by selective hydrogenation. But the activity of copper catalysts is low and the catalyst cycle time is undesirably short for the feed streams, containing higher than about 2000 ppm total alkynes due to fast deactivation caused by the deposition of polymeric material on the catalyst surface. Even though the hydrogenation may be carried out in liquid phase, some of the polymers deposited on the copper catalyst have little solubility in the liquid product stream under selective hydrogenation conditions. Due to these two reasons, the copper catalysts need improvement for the selective hydrogenation of the mixed olefin feeds, which contain relatively high concentration of total alkynes.

In general, the palladium catalysts are very active compared with the copper catalysts for selective hydrogenation of acetylenic compounds in the olefinic steams, but have lower selectivity for the acetylenes than copper-based catalysts. But the palladium catalysts exhibit low selectivity for retaining diolefins, such as 1,3-butadiene, when one is trying to remove high concentrations (>2000 ppm) of total alkynes to less than about 500 ppm total alkynes in the streams, especially when the acetylenes are reduced to less than 200 ppm. The non selectivity of palladium catalysts is not desirable in commercial practice, because it results in a loss of 1,3-butadiene. To improve olefin selectivity of palladium catalysts, silver or gold has been added to palladium catalysts in minor amounts as modifier.

U.S. Pat. No. 4,533,779 disclosed palladium/gold catalysts supported on supports such as alumina (l to l00 m²/g) for selective hydrogenation of acetylenic compounds. The contents of palladium and gold in the catalysts were in the range of 0.03 to 1 weight % and 0.003 to 0.3 weight %, respectively.

U.S. Pat. No. 4,831,200 disclosed the process for the selective hydrogenation of alkynes in olefin streams such as mixtures with 1,3-butadiene. The selective hydrogenation was carried out in two steps in sequence. In the first step, the hydrocarbon feed was passed at least partially in liquid phase with hydrogen over the palladium catalyst such as that disclosed in U.S. Pat. No. 4,533,779 discussed above. In the second step, the product stream from the first step was passed again at least partially in liquid phase with hydrogen over the copper catalyst such as that disclosed in U.S. Pat. Nos. 4,493,906 and 4,440,956 discussed above to produce significantly reduced alkyne concentration in the final product stream.

The present process has as an advantage a greater selectivity for the removal of acetylenic compounds from hydrocarbon streams with higher yields of the desired olefinic compounds than prior processes for a given catalyst. In particular, the present process provides a higher yield of 1,3-butadiene of higher purity from crude C₄ streams.

### SUMMARY OF THE INVENTION

Briefly, the present invention is a process for removal of acetylenic compounds from hydrocarbon streams comprising: contacting a hydrocarbon feed comprising a target fraction containing a first concentration of acetylenic compounds and olefins with a catalyst selective for the hydrogenation of acetylenic compounds in the presence of hydrogen and a solvent having a boiling point higher than the boiling point of the target fraction in a distillation reaction zone under reaction and distillation conditions of temperature and pressure and hydrogen concentration favoring the hydrogenation of acetylenic compounds and recovering said target fraction having a second concentration of acetylenic compounds lower than said first concentration. Preferably the solvent is selective for acetylenic compounds. Preferably the solvent is in countercurrent flow to the hydrocarbon feed in the process. The solvent may be recovered from the reactor effluent stream to recycle.

In one embodiment, the solvent may be built up in situ in the system, for example during start-up of the unit, by recycling heavy components which are produced by oligomerization and polymerization during the selective hydrogenation.

Preferably the target fraction having a second concentration of acetylenic compounds is recovered as overheads and solvent is recovered as bottoms. Using a solvent which is selective for acetylenes in the distillation/reaction mode allows the acetylenes to be stripped into the higher boiling solvent. The solvent can be the principal material contacting the hydrogenation catalyst, by adjusting the temperature and pressure conditions in the reaction/distillation column to maintain the target fraction, which has a reduced acetylene content as a result of the contact with the solvent, in the vapor phase above the catalyst bed. Limiting the exposure of the desirable unsaturated materials (olefins and dienes) to hydrogenation in the catalyst bed, reduces the collateral hydrogenation of these materials.

Although the present process is beneficially employed with any catalyst which is selective for acetylenic compounds, in a preferred mode the catalyst comprises the unsulfided metallic nickel as described above.

In practice the feed to the distillation column reactor may be vapor, mixed vapor and liquids or liquids and the feed will be below or in the lower portion of the catalyst bed. The target fraction may be the entire hydrocarbon feed or a portion thereof which comprises the acetylenes and corresponding olefins. Although, it is contemplated that the entire hydrocarbon feed will in most instances be the target fraction, i.e., a relatively discrete cut such as a C₄ cut, the feed may comprise a broader range of materials, e.g., C₂-C₇ hydrocarbons, which may or may not have corresponding unsaturated components. The lighter (C₂ and C₃) materials may exit with, for example, the C₄'s as overheads while the materials boiling in the range of the solvent exit as bottoms with the solvent.

As used herein the term “target fraction” is understood to mean that portion of the feed hydrocarbons which is being recovered as overheads after hydrotreating.

The solvent enters as a liquid above the catalyst bed and under the conditions of the distillation column reactor remains a liquid as it passes through. There is no reflux orvery minimal reflux from an overhead drum (external reflux) to the distillation column. Up to 100% of the overheads are recovered, thus the present process is carried out in the substantial absence of external reflux. The internal reflux operates to maintain the system as reaction/distillation. Preferably the external reflux is the lowest possible to maintain the system as reaction/distillation.

Hydrogen is fed to the distillation column below the catalyst bed or optionally at multiple positions along the catalytic reaction zone.

In carrying out selective hydrogenation in catalytic distillation mode in the prior art, there is always external reflux of overhead product stream from reflux drum back to the distillation column. Although the overhead reflux prolongs the catalyst service time, there is over-hydrogenation due to the reflux, resulting in a loss of valuable products such as olefins or dienes. The present invention minimizes the loss of the valuable product due to over-hydrogenation, yet prolongs the catalyst service time as the liquid solvent washes over the catalyst.

For the purposes of the present invention, the term “catalytic distillation” includes reactive distillation and any other process of concurrent reaction and fractional distillation in a column, i.e., a distillation column reactor, regardless of the designation applied thereto.

### DETAILED DESCRIPTION

Acetylenic compounds have been hydrogenated over all Group VIII metals and copper catalysts. Specifically catalytic partial hydrogenation of acetylenic compounds to olefinic compounds. All Group VIII metals (Pd, Pt, Rh, Ru, Ir and Os) and non noble metals (Fe, Co and Ni), and Cu catalysts have been known to be active for the hydrogenation of acetylenic compounds and olefins. All Group VIII noble metal catalysts and Ni catalysts have satisfactory catalytic activities for application in the commercial hydrogenation process. The present process is useful with all of the prior art catalysts heretofore used for that purpose, including those discussed above and for new catalysts as a preferred mode of operation. Preferably the present process employs the unsulfided metallic nickel or unsulfided metallic nickel modified with metallic Mo, Re, Bi or mixtures in which the catalyst is used alone or is used in combination with other acetylenic selective catalysts described above.

The solvent is determined by the boiling point of the target fraction of the hydrocarbon feed. The boiling point must be higher than the target fraction. The solvent preferably will have greater affinity for acetylenes than dienes as defined by a Henry's constant ratio of acetylene to olefin or diene of less than unity (K_{AC}/Kᵢ<1 where Kₙ=Pₙ/Xₙ; Pₙ and Xₙ=partial pressure and mole fraction of component in liquid phase, respectively). Solvents include C₄-C₁₀ paraffinic hydrocarbons, cyclohexane, methyl cyclohexane, benzene, toluene, and ethers such as tetrahydrofuran. Examples for such solvent are diethyl ether, methyl ethyl ether, dimethyl ether, ethylamine, tetrahydrofuran, acetonitrile, isopentane, benzene, cyclohexane, vinyl cyclohexene, Lean Oil 2, furfural, acyclic ketones or mixture of two or three of these.

Any satisfactory lean oil may be employed that will absorb the unsaturated organic compound. The oil may be, e.g., paraffinic or aromatic. The lean oil may, for example, be made up of compounds having elements selected from the group consisting of carbon, hydrogen, oxygen, nitrogen, halogen, and mixtures thereof and will preferably consist essentially of these elements. Of course, the lean oil may contain impurities, particularly after the process has been in operation for a period of time. Particularly preferred as lean oils are hydrocarbons which have the formula CxHy, wherein x is a number from 6 to 9 inclusive and y is a number from 6 to 18 inclusive; suitable compounds for use as lean oils include: methylcyclohexane, 2′,4,4-trimethyll-pentene, 3,4,4-trimethyl-1-pentene, 2,4,4-trimethyl-2-pentene, 3,3,4-trimethyl-1-pentene, 2,3,4-trimethyl-1-pentene, 2,3,3-trimethyl-1-pentene, 2,5-dimethylhexane, 2,4-dimethylhexane, 2,2,3-trimethylpentane, benzene, toluene, 3,4,4-trimethyl-2-pentene, 2,3,4-trimethylpentane, 2,3,3-trimethylpentane, 2,3,4-trimethyl-2-pentene, -butadiene dimer, diisobutylene, paraffins containing 8 carbon atoms, such as those obtained from an alkylation plant, amylamine, 3-chloropentane, n-butylamine, dioxane-nitro-ethane, mixtures thereof, and the like. The lean oil may, e.g., have a boiling point at standard atmospheric pressures of from about 170° F. to 320° F.

Solvents may comprise furfural with cosolvents such as ketones. Preferably at least about 3% by weight of the ketone and preferably up to about l0% by weight based on dry solvent is employed as cosolvent. The furfural comprises the principal amount of the solvent and will generally comprise from 88 to 99 weight percent of the dry solvent. Some suitable C₅-C₈ ketones are cyclopentanone, 2-methyl cyclopentanone, 3-methyl cyclopentanone, cyclohexanone, 3-methyl-2-hexanone, 4-methyl-2-hexanone, 4-methyl-3-hexanone, 5-methyl-3-hexanone, 2-heptanone, 3-heptanone, 4-heptanone, 3-ethyl-4-methyl-2-pentanone, 3,3-dimethyl-2-hexanone, 3,4-dimethyl-2-hexanone, 2,2-dimethyl-3-hexanone, 2,5-dimethyl-3′-hexanone, 4,4-dimethyl-3-hexanone, 2-methyl-4-heptanone, 3-octanone, 4-octanone. Mixtures of various suitable ketones may be employed as well as single combination with the furfural to provide the benefits of the present invention. A preferred group of ketones would be those having 6 to 7 carbon atoms. Two preferred ketones are cyclohexanone and 2-heptanone.

The catalyst must be suitably supported and spaced within the column to act as a catalytic distillation structure. Catalytic distillation structures useful for this purpose are disclosed in U.S. Pat. Nos. 4,215,011; 4,439,350; 4,443,559; 4,731,229; 5,057,468; 5,073,236; 5,189,001; 5,262,012; 5,266; 546; 5,348,710; 5,431,890; and 5,730,843 all of which are hereby incorporated by reference.

The present invention carries out the hydrogenations in a column containing one or more reaction distillation zones of catalytic distillation structures which can be appreciated to contain a vapor phase and some liquid phase as in any distillation. The distillation column reactor is operated at a pressure such that the reaction mixture (comprising the solvent and extracted acetylenes) is boiling in the bed of catalyst (distillation conditions).

The present process for olefin saturation operates at overhead pressure of said distillation column reactor in the range between 0 and 350 psig, preferably 250 or less suitable 35 to 120 psig and temperatures in said distillation reaction bottoms zone in the range of 150 to 230° F., preferably 175 to 200° F., e. g. 175 to 180° F. at the requisite hydrogen partial pressures in the range of 0.1 to less than 100 psi. The feed weight hourly space velocity (WHSV), which is herein understood to mean the unit weight of feed per hour entering the reaction distillation column per unit weight of catalyst in the catalytic distillation structures, may vary over a very wide range within the other condition parameters, e.g., 0.1 to 35 hr¹.

In the current process the temperature is controlled by operating the reactor at a given pressure to allow partial vaporization of the reaction mixture. The exothermic heat of reaction is thus dissipated by the latent heat of vaporization of the mixture. The vaporized portion (the target fraction having reduced acetylene content) is taken as overheads using little or no external reflux. The downward flowing liquid causes additional condensation within the reactor as is normal in any distillation. The contact of the condensing liquid within the column provides excellent mass transfer for dissolving the hydrogen within the reaction liquid and concurrent transfer of the reaction mixture to the catalytic sites. It is thought that this condensing mode of operation results in the excellent conversion and selectivity of the instant process and allows the lower hydrogen partial pressures and reactor temperatures noted. A further benefit that this reaction may gain from catalytic distillation is the washing effect that the internal reflux (L/D=wt. liquid just below the catalyst bed/wt. distillate) provides to the catalyst thereby reducing polymer build up and coking.

The success of catalytic distillation lies in an understanding of the principles associated with distillation. First, because the reaction is occurring concurrently with distillation, the initial reaction product (the hydrogenate acetylenes) is removed from the reaction zone as quickly as it is formed. The removal of the product minimizes oligomerization of any olefins or diolefins formed by the hydrogenation of the acetylenes. Second, because the reaction mixture is boiling, the temperature of the reaction is controlled by the boiling point of the mixture at the system pressure. The heat of the reaction simply creates more boil up, but no increase in temperature. Third, the reaction has an increased driving force because the reaction products have been removed and cannot contribute to a reverse reaction (Le Chatelier's Principle).

As a result, a great deal of control over the rate of reaction and distribution of products can be achieved by regulating the system pressure. Also, adjusting the through-put (residence time=liquid hourly space velocity) gives further control. The temperature in the reactor is determined by the boiling point of the liquid mixture present at any given pressure. The temperature in the lower portions of the column will reflect the constitution of the material in that part of the column, which will be higher than the overhead; that is, at constant pressure a change in the temperature of the system indicates a change in the composition in the column. To change the temperature the pressure is changed. Temperature control in the reaction zone is thus controlled by the pressure; by increasing the pressure, the temperature in the system is increased, and vice versa. It can also be appreciated that in catalytic distillation as in any distillation there is both a liquid phase (internal reflux) and a vapor phase. Thus, the reactants are partially in liquid phase which allows for a more dense concentration of molecules for reaction, whereas, the concurrent fractionation separates product and unreacted materials, providing the benefits of a liquid phase system (and a vapor phase system) while avoiding the detriment of having all of the components of the reaction system continually in contact with the catalyst which would limit the conversion to the equilibrium of the reaction system components.

Also, in a distillation reaction by adjustment of the pressure the location of any fraction of the material may be placed at a desired location within the column. Thus in the present process the conditions can be adjusted such that the target fraction is quickly boiled up the column after is contact with the downflow solvent, while the higher boiling solvent which contains the acetylenes can be maintained in the catalyst bed for hydrogenation.

An example of the composition of a steam cracked crude butadiene stream comprises 70 wt. % 1,3- butadiene, 10,000 wt. ppm vinyl acetylene, 2000 wt. ppm ethyl acetylene and 2000 wt. ppm methyl acetylene. In many of the prior art systems vinyl acetylene in this stream is selectively hydrogenated to about 200 wt. ppm vinyl acetylene, the loss of 1,3-butadiene due to undesired hydrogenation is already high enough. Therefore, further hydrogenation of vinyl acetylene toward completion becomes unacceptable due to the unbearable economical penalty caused by the loss of 1,3-butadiene.

The invention claimed is:

1. A process for removal of acetylenic compounds from hydrocarbon streams comprising: contacting a hydrocarbon feed comprising a target fraction containing a first concentration of acetylenic compounds and olefins with a catalyst selective for the hydrogenation of acetylenic compounds in the presence of hydrogen and a solvent having a boiling point higher than the boiling point of the target fraction in a distillation reaction zone under reaction and distillation conditions of temperature and pressure and hydrogen concentration favoring the hydrogenation of acetylenic compounds and recovering said target fraction having a second concentration of acetylenic compounds lower than said first concentration.

2. The process according to claim 1, wherein the solvent is selective for acetylenic compounds.

3. The process according to claim 1, wherein said target fraction having a second concentration of acetylenic compounds is recovered as overheads.

4. The process according to claim 1, wherein the solvent is recovered as bottoms.

5. The process according to claim 2, wherein said target fraction having a second concentration of acetylenic compounds is recovered as overheads and the solvent is recovered as bottoms.

6. The process according to claim 1, wherein said hydrocarbon feed is a liquid.

7. The process according to claim 1, wherein said hydrocarbon feed is a vapor.

8. The process according to claim 1, wherein said hydrocarbon feed is mixed liquid and vapor.

9. The process according to claim 1, wherein said solvent contacts said hydrocarbon feed in countercurrent flow.

10. The process according to claim 9, wherein solvent is downflow.

11. The process according to claim 1, having an internal reflux of lowest possible to maintain the system as reaction/distillation.

12. The process according to claim 1, wherein said solvent remains a liquid in the process.

13. The process according to claim 1, wherein said solvent is built up in situ in the system by recycling heavy components which are produced by oligomerization and polymerization during the selective hydrogenation.
